(19) United States
(12) Patent Application Publication
(10) Pub. No.: US 2026/0124501 A1
(43) Pub. Date: May 7, 2026

(54) METHOD FOR CLASSIFYING A MOVEMENT, AND A MOVEMENT CLASSIFICATION SYSTEM

(71) Applicant: Robert Bosch GmbH, Stuttgart (DE)

(72) Inventors: Jeena John, Kista (SE); Unn Dahlen, Kista (SE); Fredrik Nordstroem, Lund (SE)

(21) Appl. No.: 19/361,862

(22) Filed: Oct. 17, 2025

(30) Foreign Application Priority Data
Nov. 7, 2024 (DE) 10 2024 210 707.7

(51) Int. Cl. A63B 24/00

(52) U.S. Cl. CPC A63B 24/0062; A63B 2024/0071; A63B 2220/17; A63B 2220/30

(57) ABSTRACT

A method for classifying a movement by a movement classification system having a plurality of peripheral classification elements and a central classification unit. The peripheral classification elements are arranged on different body parts of a person, and each classifies the movement of the person based on sensor data. The method includes: receiving classification data of the plurality of peripheral classification elements using the central classification unit), wherein the classification data each include at least one peripheral classification value; classifying the movement and generating a classification value using the central classification unit based on the peripheral classification values of the plurality of peripheral classification elements; and outputting the classification value to a display unit using the central classification unit.

### CROSS REFERENCE

The present application claims the benefit under 35 U.S.C. § 119 of Germany Patent Application No. DE 10 2024 210 707.7 filed on Nov. 7, 2024, which is expressly incorporated herein by reference in its entirety.

### FIELD

The present invention relates to a method for classifying a movement and to a movement classification system.

### BACKGROUND INFORMATION

Certain methods for classifying a movement are described in the related art.

### SUMMARY

It is an object of the present invention to provide an improved method for classifying a movement and a movement classification system.

The object may be achieved by the method for classifying a movement and the movement classification system of the present invention. Advantageous embodiments of the present invention are disclosed herein.

According to one aspect of the present invention, a method is provided for classifying a movement by a movement classification system having a plurality of peripheral classification elements and a central classification unit, wherein the peripheral classification elements are arranged on different body parts of the person and each classify the movement of the person on the basis of sensor data. According to an example embodiment of the present invention, the method comprises:
receiving classification data of the plurality of peripheral classification elements by means of the central classification unit, wherein the classification data each comprise at least one peripheral classification value;
classifying the movement and generating a classification value by means of the central classification unit on the basis of the peripheral classification values of the plurality of peripheral classification elements; and
outputting the classification value to a display unit by means of the central classification unit.

A technical advantage of being able to provide an improved method for classifying a movement can thereby be achieved. The method is carried out by a movement classification system comprising a plurality of peripheral classification elements and a central classification unit.

The multiple peripheral classification elements are arranged on body parts of a moving person and are configured to classify the movement of the person on the basis of sensor values. The central classification unit is data-connected to the multiple peripheral classification elements and receives classification data from the multiple peripheral classification elements. The classification data comprise at least peripheral classification values, wherein each peripheral classification value represents the classification of the movement performed by the corresponding peripheral classification element.

According to an example embodiment of the present invention, the central classification unit creates a classification value of the movement on the basis of the multiple peripheral classification values of the multiple peripheral classification elements. The classification value of the movement is thus based on the multiple peripheral classification values of the individual peripheral classification elements. Greater precision of the classification of the movement of the person can thereby be achieved.

Because the different peripheral classification elements are arranged at different locations on the person's body, multiple movements of individual body parts of the person can be taken into account in the classification. The precision of the movement classification can thereby be improved further. The correspondingly generated classification value is then output to a display unit. This allows the display of the corresponding movement classification of the person using the movement classification system.

According to one example embodiment of the present invention, the classification value ascertained by the central classification unit matches one of the peripheral classification values provided by the plurality of peripheral classification elements if the corresponding peripheral classification value was provided by at least a predefined number of peripheral classification elements.

A technical advantage of greater precision of the classification of the movement can thereby be achieved. The classification value generated by the central classification unit matches the peripheral classification values provided by the peripheral classification elements if a predefined number of the peripheral classification elements provide the same peripheral classification value.

According to one example embodiment of the present invention, the classification data each further comprise at least one movement value, wherein the method further comprises: ascertaining a count value of cyclic partial movements of the movement on the basis of the movement values of the plurality of peripheral classification elements by means of the central classification unit; and outputting the count value to the display unit by means of the central classification unit.

A technical advantage can thereby be achieved that, in addition to the classification value, a count value of the cyclic partial movements of the movement of the person can also be provided. The multiple peripheral classification elements provide movement values in addition to the peripheral classification values. The count value created by the central classification unit is based on the movement values of the multiple peripheral classification elements, resulting in increased precision of the created count values.

According to one example embodiment of the present invention, the movement values provided by the peripheral classification elements each describe incremental movement sections of the cyclic partial movements of the respective body parts on which the peripheral classification elements are arranged, and wherein ascertaining the count value comprises:
selecting the peripheral classification elements of which the provided peripheral classification value matches the classification value of the central classification unit;
adding up the movement values provided by the peripheral classification elements at different times and generating a cumulative movement value for at least one selected peripheral classification element;
ascertaining an average movement value on the basis of the cumulative movement values of the selected peripheral classification elements if the at least one cumulative movement value of the at least one selected peripheral classification element reaches or exceeds a value of a complete cycle of the cyclic partial movement; and
increasing the count value by a value of “one” as soon as the average movement value reaches or exceeds a value of a predefined part of a complete cycle of the cyclic movement.

A technical advantage of an increased precision of the count value can thereby be achieved. For this purpose, the peripheral classification elements are first selected of which the peripheral classification values match the classification values of the central classification unit.

Thus, only the movement values of the peripheral classification elements that provided the correct classification of the movement are taken into account. The movement values provided by the multiple peripheral classification elements represent subsections of cyclic partial movements of the movement of the person.

The movement values provided by the peripheral classification elements at different times are cumulatively added up until a cumulative movement value of a peripheral classification element describes a complete cycle of the cyclic partial movement.

Based on this, an average movement value of all movement values of the selected peripheral classification elements is ascertained.

As soon as the average movement value of all movement values of the selected peripheral classification elements describes a complete cycle of a cyclic movement, the counter is increased by the numerical value 1. This allows extremely precise ascertainment of the count value of the cyclic movement.

On the one hand, only the peripheral classification elements are taken into account in the calculation of the count value. On the other hand, the different partial movements of the different body parts are taken into account in the average movement value.

According to one example embodiment of the present invention, the method further comprises:
ascertaining a success value for a peripheral classification element if the peripheral classification value provided by the particular peripheral classification element matches the classification value ascertained by the central classification unit; and
evaluating a performance of the plurality of peripheral classification elements by means of the central classification unit on the basis of the success values.

A technical advantage can thereby be achieved that a performance of the classification by the individual peripheral classification elements can be ascertained. The correspondingly generated performance can be presented to the user.

Alternatively or additionally, the performance can be taken into account in further classification steps by disregarding poorly performing peripheral classification elements in further steps.

According to one example embodiment of the present invention, ascertaining the count value also comprises:
ascertaining a movement speed of the cyclic partial movements ascertained by the peripheral classification elements on the basis of the movement values provided by the peripheral classification elements and on the basis of time stamps provided by the peripheral classification elements for the respective movement values and generating movement values by means of the central classification unit, wherein the time stamps define times at which the movement values were ascertained by the peripheral classification elements.

A technical advantage can thereby be achieved of being able to ascertain a movement speed of the cyclic partial movements on the basis of the movement values of the various peripheral classification elements. This can again be displayed to the user as additional information.

According to one example embodiment of the present invention, the peripheral classification values and/or the movement values of a peripheral classification element of the plurality of peripheral classification elements remain disregarded for the ascertainment of the classification value and/or for the ascertainment of the count value if a time period between peripheral classification values and/or movement values provided successively by the same peripheral classification element exceeds a predefined limit value.

A technical advantage of making the ascertainment of the classification value and/or of the count value more precise can thereby be achieved. Peripheral classification elements that have not transmitted any new classification data to the central classification unit for longer than a predetermined time period are disregarded in the further ascertainment of the classification or the count value, and the peripheral classification values or movement values already transmitted remain disregarded in further steps.

Faulty peripheral classification elements or peripheral classification elements located on body parts that do not perform any cyclic movements within the time can thereby be excluded from the movement classification.

According to one example embodiment of the present invention, the method further comprises:
ascertaining a pause in the movement by means of the central classification unit if the number of peripheral classification elements that have provided a peripheral classification value matching the classification value ascertained by the central classification unit falls below the predefined number, and the time period between peripheral classification values provided successively by at least one peripheral classification element reaches or falls below the predefined time period.

A technical advantage can thereby be achieved that a pause in the movement of the user can be detected precisely. This is detected as soon as fewer than the predefined number of peripheral classification elements provide a peripheral classification value that matches the classification value of the central classification unit, but at the same time the peripheral classification elements provide classification data to the central classification unit within a shorter time interval than the predefined time period.

The predefined time period describes a maximum time period that may elapse between the provision by a peripheral classification element of two successive classification data before the peripheral classification element in question is evaluated as inactive.

This means that the peripheral classification elements continue to provide classification data at time intervals shorter than the predefined time period, which in turn indicates that the movement classification system is still operating.

Because the pause in the movement is identified, the classification values and count values already generated are retained, and the movement classification system thus waits for the user to continue the movement. However, a complete reset of the system does not occur and the above method can be continued with the values already generated when the movement is resumed.

According to one example embodiment of the present invention, the method further comprises:
ascertaining that the classification system is in an idle state if the number of peripheral classification elements that have provided a peripheral classification value matching the classification value ascertained by the central classification unit falls below the predefined number, and the time period between peripheral classification values provided successively by at least one peripheral classification element exceeds the predefined time period.

A technical advantage of providing a precise ascertainment of an idle state of the movement classification system can thereby be achieved.

This occurs as soon as the number of peripheral classification elements that provide a peripheral classification value matching the classification value of the classification unit is less than the predefined number, and a time period greater than the predefined time period has elapsed since the peripheral classification elements last provided classification data. This indicates that the system is in the idle state.

According to one example embodiment of the present invention, the method further comprises:
resetting the classification value and/or the count value and/or the speed value and/or the success values by means of the central classification unit if the idle state of the classification system has been ascertained.

A technical advantage can thereby be achieved that the movement classification system can create a precise classification of the movement when used again without influencing the previous classification by resetting the above values when the classification system is in the idle state.

According to one example embodiment of the present invention, the classified movement is one from the following list: walking, running, hiking, mountain climbing, swimming, cross-country skiing, cycling, and wherein the count value relates to a cyclic partial movement from the following list: step, swimming arm stroke, swimming leg kick, cycling crank revolution.

A technical advantage can thereby be achieved that the method according to the present invention can be used to classify a wide variety of movements with cyclic partial movements.

According to one aspect of the present invention, a movement classification system is provided having a central classification unit and a plurality of peripheral classification elements, wherein the peripheral classification elements each comprise at least one movement sensor, and wherein the movement classification system is configured to carry out the method for classifying a movement according to one of the above-described embodiments.

A technical advantage can thereby be achieved that an improved movement classification system is provided, which is configured to carry out the method for classifying a movement with the aforementioned technical advantages.

According to one example embodiment of the present invention, the peripheral classification elements and/or the central classification unit are designed as wearables.

A technical advantage can thereby be achieved that the classification system can be easily worn by a person and thus the movements of the person can be classified by the movement classification system.

According to one aspect of the present invention, a computing unit is provided, which is configured to carry out the method for classifying a movement according to one of the above-described embodiments.

According to one aspect of the present invention, a computer program product is provided, comprising commands that, when the program is executed by a data processing unit, cause the data processing unit to execute the method for classifying a movement according to one of the above-described embodiments.

Embodiments of the present invention are described with reference to the figures.

### BRIEF DESCRIPTION OF THE DRAWINGS

FIG. 1 shows a schematic representation of a movement classification system according to one example embodiment of the present invention.

FIG. 2 shows a flowchart of a method for classifying a movement according to one example embodiment of the present invention.

FIG. 3 shows a further flowchart of the method for classifying a movement according to a further example embodiment of the present invention.

FIG. 4 shows a further flowchart of the method for classifying a movement according to a further example embodiment of the present invention.

FIG. 5 shows a further flowchart of the method for classifying a movement according to a further example embodiment of the present invention.

FIG. 6 shows a further flowchart of the method for classifying a movement according to a further example embodiment of the present invention.

FIG. 7 shows a schematic representation of a computer program product, according to an example embodiment of the present invention.

### DETAILED DESCRIPTION OF EXAMPLE EMBODIMENTS

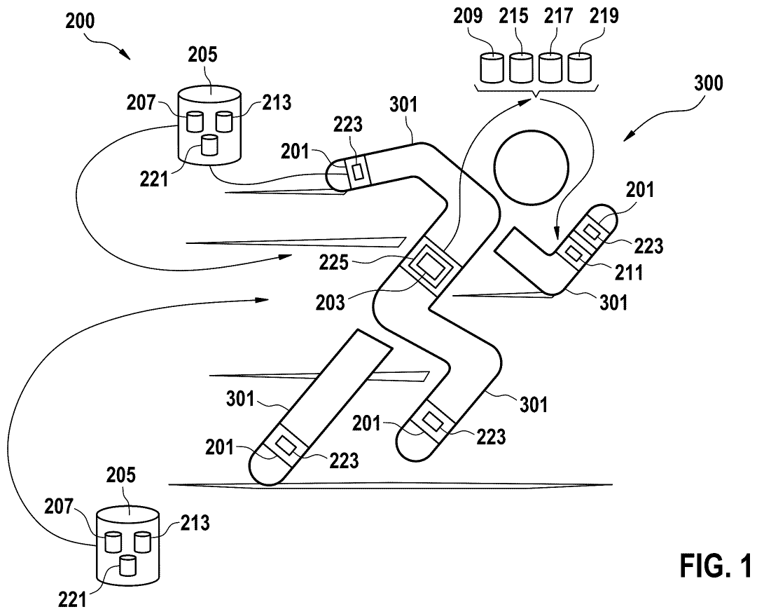

FIG. 1 is a schematic representation of a movement classification system 200 according to one embodiment.

FIG. 1 shows a user 300 with a movement classification system 200 according to the present invention. In the embodiment shown, the movement classification system 200 comprises four peripheral classification elements 201, which are each positioned on the four extremities of the person 300.

Furthermore, the movement classification system 200 comprises a central classification unit 203, which in the embodiment shown is positioned on the torso of the person 300.

The multiple peripheral classification elements 201 each comprise at least one movement sensor 223, via which the movements of the person 300 can be ascertained. The movement sensors 223 can be designed, for example, as acceleration sensors, gyroscopic sensors or other conventional movement sensors.

Using the sensor values of the movement sensors 223, the peripheral classification elements 201 are configured to independently classify the movement of the person 300 and to generate peripheral classification values 207.

The peripheral classification values 207 describe the classification of the movement of the person 300 that was independently generated by the particular peripheral classification element 201.

The various peripheral classification elements 201 are data-connected to the central classification unit 203 and send classification data 205 comprising at least the peripheral classification values 207 to the central classification unit 203.

On the basis of the peripheral classification values 207, the central classification unit 203 generates a classification value 209. The classification value 209 represents a classification of the movement of the person 300 and is based on the peripheral classification values 207 of the peripheral classification elements 201.

According to one embodiment, the central classification unit 203 generates the classification value 209 in that the classification unit 203 adopts the peripheral classification values 207 transmitted by the peripheral classification elements 201, provided that the number of peripheral classification elements 201 that have transmitted the same peripheral classification value 207 reaches or exceeds a predefined limit value.

In the embodiment shown, the user 300 is shown in a running movement. Furthermore, the person 300 has a peripheral classification element 201 on both arms and both legs.

The central classification unit 203 can now be configured to adopt the peripheral classification value 205 as the classification value 209, provided that at least two of the peripheral classification elements 201 provide the same peripheral classification value 207.

For example, if at least one peripheral classification element 201 on an arm and one peripheral classification element 201 on a leg of the person 300 classifies the movement of the person 300 as a running movement, the central classification unit 203 will also classify the movement of the person 300 as running.

According to one embodiment, in addition to the peripheral classification values 205, movement values 213 are also transmitted by the peripheral classification elements 201 as part of the classification data 205 to the central classification unit 203.

On the basis of the movement values 213 of the peripheral classification elements 205, the central classification unit 203 generates a count value 215 of the movement of the person 300.

The movement values 213 of the peripheral classification elements 201 describe movement sections of cyclic partial movements of the movement of the person 300. In the embodiment shown, the cyclical partial movements describe the back-and-forth swinging of the arms and the stepping movements of the legs of the person 300.

The count value 215, which by the central classification unit 203 on the basis of the movement values 213, quantifies the successive execution of the cyclic partial movements. In the example shown, the count value 215 can, for example, describe the number of steps taken during the running movement.

According to one embodiment, the peripheral classification elements 201 further transmit time stamps 221 as part of the classification data 205 to the central classification unit 203. The time stamps 221 describe times at which the movement values 213 were recorded.

On the basis of the time stamps 221 and the movement values 213, the central classification unit 203 generates a speed value 219.

The speed value 219 describes the execution speed of the cyclic partial movements, which are represented by the movement values 213 of the peripheral classification elements 201.

According to one embodiment, the central classification unit 203 further generates a success value 217. The success value 217 is generated for each peripheral classification element 201 each time the peripheral classification element 201 provides a peripheral classification value 207 that matches the classification value 209 generated by the central classification unit 203.

According to the present invention, the individual peripheral classification elements 201 continuously carry out classifications of the movement during the movement of the user 300 and provide classification data 205 including at least the peripheral classification value 207 and optionally additionally the movement value 213 and the time stamp 221.

The peripheral classification elements 201 provide the classification data 205 to the central classification unit 203 at their own timing.

The individual peripheral classification elements 201 in particular provide the classification data 205 when it was possible to carry out a successful classification of the movement.

At irregular time intervals, classification data 205 are thus received by the central classification unit 203 from the individual peripheral classification elements 201.

Each time a peripheral classification value 207 of a peripheral classification element 201 matches the previously generated classification value 209 of the central classification unit 203, a corresponding success value 217 is assigned to this peripheral classification element 201. The corresponding success values 217 can be used to assess the performance of the various peripheral classification elements 201.

In the embodiment shown, the movement classification system 200 further comprises a display unit 211. After generation of the classification value 209 and/or the count value 215 and/or the speed value 219 and/or the success value 217, these values are provided by the central classification unit 203 to the display unit 211 and can be displayed to the user 300 via this unit.

In the embodiment shown, the peripheral classification elements 201 and the central classification unit 203 are designed as wearables and can be fixed at various locations on the body of the user 300. The central classification unit 203 is executed on a computing unit 225 of the movement classification system 200.

Alternatively, the central classification unit 203 can also be executed on an external computing unit 225, for example a smartphone, tablet or a PC.

In addition to the running movement shown here, the method according to the present invention and the movement classification system 200 according to the present invention can also be used to classify walking movements, hiking movements, mountain climbing movements, swimming movements, cross-country skiing movements, cycling movements or other movements that comprise cyclical partial movements.

The count value 215 can represent a numerical value of a step movement, a swimming arm stroke movement, a wheel crank rotation movement or other cyclic partial movements.

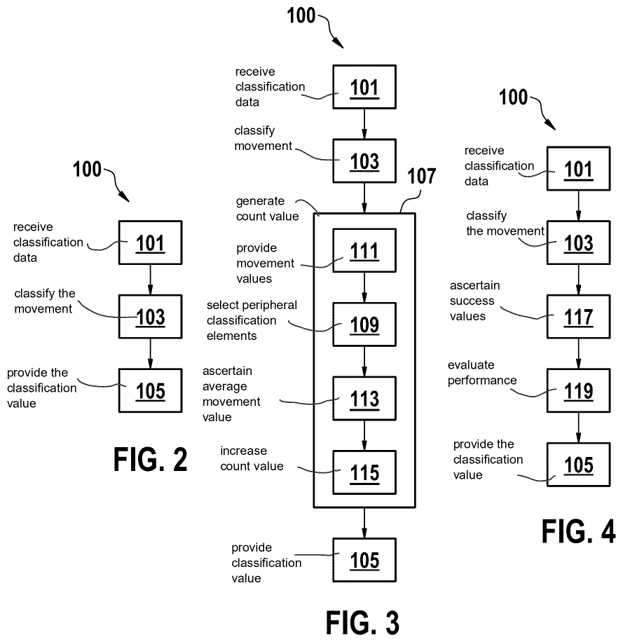

FIG. 2 is a flowchart of a method 100 for classifying a movement according to one embodiment.

In order to classify the movement of the user, in a first method step 101, the classification data 205 of the multiple peripheral classification elements 201 are received by the central classification unit 203.

In a further method step 103, the central classification unit 203 classifies the movement on the basis of the peripheral classification values 207 of the plurality of peripheral classification elements 201 and generates the classification value 209.

In a further method step 105, the classification value 209 is provided by the central classification unit 203 to the display unit 211.

FIG. 3 is a further flowchart of the method 100 for classifying a movement according to a further embodiment.

The embodiment in FIG. 3 is based on the embodiment in FIG. 2 and comprises all the method steps described there.

In the embodiment shown, the classification data 205 provided by the peripheral classification elements 201 comprise, in addition to the parallel peripheral classification values 207, the movement values 213.

In a method step 107, the count value 215 is generated by the central classification unit 203 on the basis of the movement values 213 of the peripheral classification elements 201.

In a further method step 111, the movement values 213 provided by the peripheral classification elements 201 at different times are added up and a cumulative movement value 213 is ascertained for at least one selected peripheral classification element 201.

$${θ}_{i , k} = {θ}_{i , k - 1} + {Δθ}_{i , k}$$

Here, θ_{i,k} is the k-th movement value 213 of the i-th peripheral classification element 201, θ_{i,k-1} is the (k−1)-th movement value 213 of the i-th peripheral classification element 201, and Δθ_{i,k} is the change in the movement from the (k−1)-th movement value 213 to the immediately following k-th movement value 213, where:

$${Δθ}_{i} = {Δθ}_{i} + d {θ}_{i}$$

$$d {θ}_{i} = max ( P , ( {θ}_{i , k} - {θ}_{i , k - 1} ) )$$

For this purpose, in a method step 109, the peripheral classification elements 201 are selected of which the provided peripheral classification value 207 matches the classification value 209 of the central classification unit 203.

The movement values 213 of the peripheral classification elements 201 describe cyclic movements of the body parts on which the peripheral classification elements 201 are positioned. The movement values 213 can describe incremental movement sections of the cyclic movements. For example, the peripheral classification elements 201 can be configured to record a predefined number of movement values 213 per full cycle of the cyclic movement.

The movement values 213 can be expressed in the unit radian, in which a complete cycle is expressed by 2π. The movement value can also be expressed in percent or degrees.

The movement values 213 can further comprise phase information. Using the phase information, movements of the individual body parts on which the respective peripheral classification elements 201 are arranged can be related to one another.

For example, it can be ascertained that movements are carried out in phase or antiphase to one another. For example, during walking or running, the swinging in phase of the right arm with the left leg and of the left arm with the right leg can be detected.

In method step 109, the peripheral classification elements 201 are selected of which the movement values 213 describe such movements executed in phase.

In a further method step 113, an average movement value is ascertained on the basis of the cumulative movement values 213 of the selected peripheral classification elements 201 if the at least one cumulative movement value of the at least one selected peripheral classification element 201 reaches or exceeds a value of a complete cycle of the cyclic partial movement.

The average movement value is calculated as a weighted average over a set of peripheral classification elements 201 as follows:

$$\overline{Δθ} = {\sum }_{i ∈ I} {ω}_{i} {Δθ}_{i}$$

where Δ̅θ̅ is the average movement value, Δθᵢ is the cumulative movement value 213 of the i-th peripheral classification element 201, and ωᵢ is the weighting value associated with the i-th peripheral classification element 201. The set I is a set of all the peripheral classification elements 201 that were used to calculate the average movement value. For this purpose, for example, primarily peripheral classification elements 201 with a high performance can be selected.

$$I = \{ i : {a}_{i} = m , {Δθ}_{i} > c * max ( Δθ ) \}$$

where aᵢ is the peripheral classification value 207 of the i-th peripheral classification element 201, m is the classification value 209 of the central classification unit 203, Δθᵢ is the cumulative movement value 213 of the i-th peripheral classification element 201, and 40 is a vector representation of all the cumulative movement values 213 of the selected peripheral classification elements 201.

The weighting value ωᵢ can be scaled with the performance of the respective peripheral classification elements 201:

$${ω}_{i} = 1 / {n}_{I}$$

where n_{I} represents the size of the set.

The movement values 213 of the peripheral classification elements 201 describe movement sections of the cyclic movements, in particular of the individual body parts of the user 300.

By adding up the movement values 213 provided successively by the various peripheral classification elements 201, a predefined part of a complete cycle of the cyclic movement ascertained by the particular peripheral classification element 201 is achieved for each selected peripheral classification element 201 after a certain number of successively provided movement values 213 has been added up. The predefined part can be set to a quarter, half or three quarters of a cycle. Other values are of course also possible.

The peripheral classification elements 201 can be configured such that the movement values 213 are recorded in a predetermined number of incremental measuring steps within the cycles of the cyclic movements.

For example, for each of the various peripheral classification elements 201, ten movement values 213 can be recorded for each cycle of the cyclic movement.

The ten movement values 213 are recorded successively at different times and transmitted to the central classification unit 203. After the ten successively recorded movement values have been added up, a complete cycle of the cyclic movement is achieved.

The average movement value is calculated on the basis of all the cumulative movement values of the selected peripheral classification elements 201.

Since current movement values 213 of the peripheral classification elements 201 are provided constantly during ongoing movement, and accordingly the cumulative movement values 213 of the respective peripheral classification elements 201 increase over time, the average movement value will also assume a constantly larger value as time progresses.

In a further method step 115, the count value 215 is subsequently increased by a numerical value of 1 as soon as the average movement value reaches or exceeds a predefined part, i.e., a multiple, of a complete cycle of the cyclic movement.

The peripheral classification elements 201 selected for calculating the count value 215 can change over time depending on the correspondingly provided peripheral classification values 207; in particular, the number of selected peripheral classification elements 201 can vary over time on the basis of the provided peripheral classification values 207.

By calculating the average movement value, a precise ascertainment of the count value 215 can be achieved.

FIG. 4 is a further flowchart of the method 100 for classifying a movement according to a further embodiment.

The embodiment in FIG. 4 is based on the embodiment in FIG. 2 and comprises all the method steps described there.

In the embodiment shown, in a method step 117, the success values 217 for the peripheral classification elements 201 are ascertained if the peripheral classification values 207 provided by the respective peripheral classification elements 201 match the classification values 209 ascertained by the central classification unit 203.

On the basis of the success values 217, feedback fᵢ can be calculated:

$${f}_{i} = \frac{{s}_{i}}{expected success}$$

where si is the success value 217 of the i-th peripheral classification element 201, and where the expected success can be ascertained taking into account the peripheral classification element 201 with the highest success values.

In a further method step 119, the central classification unit 203 evaluates a performance of the plurality of peripheral classification elements 201 on the basis of the success values 217.

The particular peripheral classification element 201 receives a higher performance value with a higher number of success values 217 and a correspondingly lower performance value with a lower number of achieved success values 217.

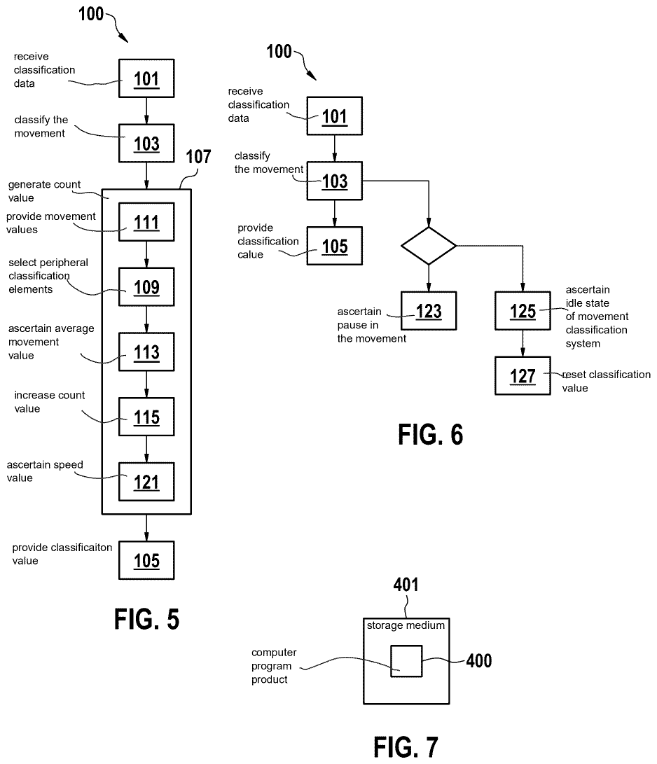

FIG. 5 is a further flowchart of the method 100 for classifying a movement according to a further embodiment.

The embodiment in FIG. 5 is based on the embodiment in FIG. 3 and comprises all the method steps described there. In the embodiment shown, in a method step 121, the speed value 219 of the cyclic movement is ascertained by the central classification unit 203 on the basis of the movement values 213 and the time stamps 221.

The corresponding movement values 213 and/or speed values 219 can be provided to the display unit 211 in method step 105.

FIG. 6 is a further flowchart of the method 100 for classifying a movement according to a further embodiment.

The embodiment in FIG. 6 is based on the embodiment in FIG. 2 and comprises all the method steps described there.

In a method step 123, a pause in the movement of the user 300 is ascertained if the number of peripheral classification elements 201 of which the peripheral classification values 207 match the classification value 209 of the central classification unit 203 falls below the predefined number, and if the time period between the successively provided peripheral classification values 207 reaches or falls below the predefined time period.

In a method step 125, however, the idle state of the movement classification system 200 is ascertained by the central classification unit 201 if, in turn, the number of peripheral classification elements 201 that provide a peripheral classification value 207 matching the classification value 209 is smaller than the predefined number, and if the time period between peripheral classification values 207 provided immediately successively exceeds the predefined time period.

If the predefined time period is exceeded, it is assumed that no movement of the person 300 was ascertained by the movement classification system 200 for a time period that is too long for this to be explained by a pause between movements of the person 300.

If the last transmission of peripheral classification values 207 occurred longer ago than the predefined time period, this is instead identified with the idle state of the movement classification system 200.

In a further method step 127, the central classification unit 203 resets the classification value 209 and/or the count value 215 and/or the speed value 219 and/or the success values 217 that were generated during the classification of the earlier movement, if the idle state of the movement classification system 200 was ascertained.

The embodiments of the method 100 of FIGS. 2 to 6 can also be combined with one another in a manner different from the embodiments described above.

FIG. 7 is a schematic representation of a computer program product 400, comprising commands that, when the program is executed by a data processing unit, cause the data processing unit to carry out the method 100 for classifying a movement.

In the embodiment shown, the computer program product 400 is stored on a storage medium 401. Here, the storage medium 401 can be any storage medium from the prior and related art.

What is claimed is:

1. A method for classifying a movement by a movement classification system having a plurality of peripheral classification elements and a central classification unit, wherein the peripheral classification elements are arranged on different body parts of a person and each classifies the movement of the person based on sensor data, the method comprising the following steps:
receiving classification data of the plurality of peripheral classification elements using the central classification unit, wherein the classification data each include at least one peripheral classification value;
classifying the movement and generating a classification value using the central classification unit based on the peripheral classification values of the plurality of peripheral classification elements; and
outputting the classification value to a display unit using the central classification unit.

2. The method according to claim 1, wherein the classification value ascertained by the central classification unit matches one of the peripheral classification values provided by the plurality of peripheral classification elements when the the one peripheral classification value was provided by at least a predefined number of the peripheral classification elements.

3. The method according to claim 1, wherein the classification data each further includes at least one movement value, and wherein the method further comprises:
ascertaining a count value of cyclic partial movements of the movement based the movement values of the plurality of peripheral classification elements using the central classification unit; and
outputting the count value to the display unit using the central classification unit.

4. The method according to claim 3, wherein the movement values provided by the peripheral classification elements each describe incremental movement sections of the cyclic partial movements of respective body parts on which the peripheral classification elements are arranged, and wherein the ascertaining of the count value includes:
selecting the peripheral classification elements of which the provided peripheral classification value matches the classification value of the central classification unit;
adding up the movement values provided by the peripheral classification elements at different times and generating a cumulative movement value for at least one selected peripheral classification element;
ascertaining an average movement value based on the cumulative movement values of the selected peripheral classification elements when the at least one cumulative movement value of the at least one selected peripheral classification element reaches or exceeds a value of a complete cycle of the cyclic partial movement; and
increasing the count value by a value of “one” as soon as the average movement value reaches or exceeds a value of a complete cycle of a cyclic movement.

5. The method according to claim 1, further comprising:
ascertaining a success value for a peripheral classification element when the peripheral classification value provided by the peripheral classification element matches the classification value ascertained by the central classification unit; and
evaluating a performance of the plurality of peripheral classification elements using the central classification unit based on the success values.

6. The method according to claim 3, wherein ascertaining (107) further comprises:
ascertaining a movement speed of the cyclic partial movements ascertained by the peripheral classification elements based on the movement values provided by the peripheral classification elements and based on time stamps provided by the peripheral classification elements for the movement values, and generating speed values using the central classification unit, wherein the time stamps define times at which the movement values were ascertained by the peripheral classification elements.

7. The method according to claim 3, wherein the peripheral classification values and/or the movement values of a peripheral classification element of the plurality of peripheral classification elements are disregarded for the ascertainment of the classification value and/or for the ascertainment of the count value, when a time period between peripheral classification values and/or movement values provided successively by the same peripheral classification element exceeds a predefined limit value.

8. The method according to claim 1, further comprising:
ascertaining a pause in the movement using the central classification unit when a number of the peripheral classification elements that have provided a peripheral classification value matching the classification value ascertained by the central classification unit falls below a predefined number, and a time period between peripheral classification values provided successively by at least one peripheral classification element reaches or falls below a predefined time period.

9. The method according to claim 3, further comprising:
ascertaining that the movement classification system is in an idle state when a number of the peripheral classification elements that have provided a peripheral classification value matching the classification value ascertained by the central classification unit falls below the predefined number, and a time period between peripheral classification values provided successively by at least one peripheral classification element exceeds a predefined time period.

10. The method according to claim 9, further comprising:
resetting the classification value and/or the count value and/or a speed value and/or success values using the central classification unit when the idle state of the movement classification system has been ascertained.

11. The method according to claim 3, wherein the classified movement is one from the following list: walking, running, hiking, mountain climbing, swimming, cross-country skiing, cycling, and wherein the count value relates to a cyclic partial movement from the following list: step, swimming arm stroke, swimming leg kick, cycling crank revolution.

12. A movement classification system, comprising:
a central classification unit; and
a plurality of peripheral classification elements, wherein the peripheral classification elements each include at least one movement sensor;
wherein the movement classification system is configured to classifying a movement including by:
receiving classification data of the plurality of peripheral classification elements using the central classification unit, wherein the classification data each include at least one peripheral classification value,
classifying the movement and generating a classification value using the central classification unit based on the peripheral classification values of the plurality of peripheral classification elements, and
outputting the classification value to a display unit using the central classification unit.

13. The movement classification system according to claim 12, wherein the peripheral classification elements and/or the central classification unit are wearables.

14. A computing unit configured to classifying a movement by a movement classification system having a plurality of peripheral classification elements and a central classification unit, wherein the peripheral classification elements are arranged on different body parts of a person and each classifies the movement of the person based on sensor data, the computing unit configured to perform the following steps comprising:
receiving classification data of the plurality of peripheral classification elements using the central classification unit, wherein the classification data each include at least one peripheral classification value;
classifying the movement and generating a classification value using the central classification unit based on the peripheral classification values of the plurality of peripheral classification elements; and
outputting the classification value to a display unit using the central classification unit.

15. A non-transitory computer-readable medium on which is stored a computer program product including commands for classifying a movement by a movement classification system having a plurality of peripheral classification elements and a central classification unit, wherein the peripheral classification elements are arranged on different body parts of a person and each classifies the movement of the person based on sensor data, the commands, when executed by a data processor, causing the data processor to perform the following steps:
receiving classification data of the plurality of peripheral classification elements using the central classification unit, wherein the classification data each include at least one peripheral classification value;
classifying the movement and generating a classification value using the central classification unit based on the peripheral classification values of the plurality of peripheral classification elements; and
outputting the classification value to a display unit using the central classification unit.
